(19) United States
(12) Patent Application Publication
(10) Pub. No.: US 2016/0015474 A1
(43) Pub. Date: Jan. 21, 2016

(54) SURGICAL INSTRUMENT TRACKING ASSEMBLY

(71) Applicant: Claronav Inc., North York (CA)

(72) Inventor: Doron Dekel, Toronto (CA)

(21) Appl. No.: 14/800,225

(22) Filed: Jul. 15, 2015

Related U.S. Application Data
(60) Provisional application No. 62/025,206, filed on Jul. 16, 2014.

(51) Int. Cl. A61B 19/00

(52) U.S. Cl. CPC A61B 19/54; A61B 19/5244; A61B 2019/5255; A61B 2019/5437

(57) ABSTRACT

A surgical instrument tracking assembly includes a trackable target and an adapter that can be rigidly coupled to the trackable target such that the pose of the adapter can be determined from the pose of the trackable target. The tracking assembly also includes stretchable tape for stretchably winding around the adapter and a surgical instrument to provide a tensile clamping force holding the adapter to the surgical instrument in a fixed, stable, spatial relationship so that a pose of the surgical instrument is determinable from the pose of the adapter. The stretchable tape may be highly elastic to provide a long-lasting tensile clamping force, sufficiently strong to resist tearing when being stretched by a human hand, and self-engaging such that once the tape is wound on itself, the long-lasting tensile clamping force of the winding is maintained even when the pulling force is removed.

### PRIORITY

This application claims the benefit of U.S. Provisional Patent Application No. 62/025,206, filed Jul. 16, 2014, the entirety of which is incorporated herein by reference.

### FIELD

The described embodiments relate to the field of surgical instruments. In particular, the teachings described herein relate to tracking assemblies for surgical instruments.

### BACKGROUND

During surgery, it is often useful to track the position of surgical instruments being used. This tracking may be used to visually display the position and orientation of a surgical instrument to a surgeon or dentist when the instrument is positioned near to, or within, the patient. For example, dental navigation systems may track the position of a drill inserted in a dental handpiece relative to a patient's jaw. Examples of dental navigation systems include those developed by Robodent (www.robodent.de) and DenX (www.denx.com). These systems and other surgical navigation systems can be used to track the position and orientation of a surgical instrument in real time.

The real-time computerized measurement of spatial position and orientation (pose) of specially marked objects moving unpredictably through an unstructured scene is of great interest in the surgical field. The pose of the surgical instrument and the patient can be tracked separately so that the real-time location of the instruments relative to the patient's anatomy can be acquired and displayed.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

In accordance with an embodiment, there is provided a surgical instrument tracking assembly. The surgical instrument tracking assembly can include a trackable assembly having a trackable target, the trackable target being trackable by a tracking device to determine a pose of the trackable target, and an adapter rigidly coupleable to the trackable target such that a pose of the adapter is determinable from the pose of the trackable target. The surgical instrument tracking assembly can also include a length of stretchable tape for stretchably winding around the adapter and the surgical instrument to provide a tensile clamping force holding the adapter to the surgical instrument in a fixed, stable, spatial relationship, where the adapter and trackable target can be subsequently detachable by removing the stretchable tape. The pose of the surgical instrument may be determinable from the pose of the adapter and from the pose of the trackable target when the trackable target is attached to the adapter. The length of stretchable tape can be configured to be i) highly elastic to provide a long-lasting tensile clamping force for holding the adapter against the surgical instrument, ii) sufficiently strong to resist tearing when being stretched by a pulling force of one human hand as the length of stretchable tape is wound around the adapter and the surgical instrument, and iii) self-engaging such that once the tape is wound on itself while being stretched by the pulling force, the long-lasting tensile clamping force of the winding can be maintained even when the pulling force is removed.

In some embodiments, the tape may be self-fusing silicone tape.

In some embodiments, the surgical instrument may be a dental handpiece.

In some embodiments, the surgical instrument tracking assembly can also include sticky putty that can be applied between the adapter and the surgical instrument prior to winding the length of stretchable tape around the adapter and the surgical instrument.

In some embodiments, the surgical instrument tracking assembly can also include a rubber cover sleeve configured to engage the adapter and the surgical instrument when attached together to cover the length of stretchable tape.

In some embodiments, the adapter may have a plurality of possible contact regions spaced from each other and configured such that the adapter is orientable relative to the surgical instrument such that at least three contact regions in the plurality of possible contact regions are in contact with the surgical instrument.

In some embodiments, the surgical instrument may have a substantially cylindrical circumferential surface and the at least three contact regions can be oriented to contact the substantially cylindrical surface of the surgical instrument.

In some embodiments, the at least three contact regions may include at least four contact regions for contacting the substantially cylindrical surface of the surgical instrument.

In some embodiments, the adapter can include a plurality of arms and the plurality of possible contact regions can include a plurality of arm contact regions. Each arm of the plurality of arms can include a corresponding arm contact point in the plurality of arm contact regions.

In some embodiments, the adapter may have a central portion defining a radius of curvature, and the plurality of arms may include two side arms extending from the central portion, and a forward arm extending from the central portion in a forward direction substantially orthogonal to a direction of the radius of curvature where the central portion and the two side arms may together define a substantially concave shape. The plurality of possible contact regions may include the central contact point, with the central portion including the central contact point.

In some embodiments, the plurality of arms can include four arms and the plurality of arm contact regions can include four arm contact regions, with a corresponding arm contact point for each arm of the four arms, such that the four arm contact regions are orientable to simultaneously contact the substantially cylindrical surface of the surgical instrument.

In some embodiments, the substantially concave shape may define a diameter of about 20 mm.

In some embodiments, the substantially cylindrical circumferential surface of the surgical instrument can be tapered such that a diameter of surgical instrument varies along at least a portion of a length of the surgical instrument to provide a substantially frustro-conical circumferential surface.

In some embodiments, the trackable target may define an optically detectable pattern detectable by the tracking device to determine the pose of the trackable target, where the optically detectable pattern includes a plurality of high contrast regions.

In some embodiments, the adapter can be constructed from a corrosive resistant strong material selected from the group comprising stainless steel and engineered plastic.

In some embodiments, the adapter may include a rigid portion and at least one deformable portion, the at least one deformable portion can be configured to conform to a surface of the surgical instrument when pressed against that surface by the tensile clamping force of the stretchable tape, and the at least one deformable portion can include at least one of the possible contact regions. In some embodiments, the rigid portion and the at least one deformable portion can be constructed from the same material and the rigid portion of the adapter can be thicker than each deformable portion.

In some embodiments, the trackable assembly may further include at least one coupler for rigidly coupling the adapter and the trackable target together such that a pose of the adapter is determinable from the pose of the trackable target. In some embodiments, the at least one coupler for rigidly coupling the adapter and the trackable target may be mounted on at least one of the trackable target and the adapter, the at least one coupler can be releasable to detach the trackable target from the adapter.

In some embodiments, the adapter and the trackable target may be formed integrally.

In some embodiments, the adapter can be constructed to withstand repeated steam-sterilization cycles comprising high pressure steam up to 135° C. for 15 minutes.

In some embodiments, the length of stretchable tape may be heat resistant to withstand repeated cycles of steam sterilization comprising high pressure steam up to 135° C. for 15 minutes.

### BRIEF DESCRIPTION OF THE DRAWINGS

Several embodiments will now be described in detail with reference to the drawings, in which:

FIG. 1 shows a pair of perspective views of a surgical instrument tracking assembly in accordance with an embodiment;

FIG. 2 shows a side view of an adapter held against a surgical instrument in accordance with an embodiment;

FIG. 3A is a top view illustrating components of the surgical instrument tracking assembly of FIG. 1 in accordance with an embodiment;

FIG. 3B is a perspective view illustrating the example adapter component of FIG. 3A in accordance with an embodiment;

FIG. 3C is a side view illustrating the adapter of FIG. 3B positioned to contact a surgical instrument in accordance with an embodiment;

FIG. 3D is a side view illustrating the adapter and surgical instrument of FIG. 3C after a single winding of stretchable tape has been applied around the adapter and the surgical instrument in accordance with an embodiment;

FIG. 3E is a side view illustrating the adapter and surgical instrument of FIG. 3C after multiple windings of stretchable tape have been applied around the adapter and the surgical instrument in accordance with an embodiment;

FIG. 3F is a perspective view illustrating the surgical instrument tracking assembly components of FIG. 3E with the adapter detached from the surgical instrument in accordance with an embodiment;

FIG. 4 is a perspective view of another example adapter positioned to contact a surgical instrument in accordance with an embodiment;

FIG. 5 is a perspective view of another example of a surgical instrument tracking assembly using the adapter of FIG. 4 in accordance with an embodiment;

FIG. 6 is a perspective view of the example adapter of FIG. 4 held against a surgical instrument in accordance with an embodiment;

FIG. 7 is a perspective view of a rubber cover sleeve positioned to cover an adapter and a surgical instrument in accordance with an embodiment.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that the terms “coupled” and “coupleable” used herein indicates that two elements can be directly coupled to one another or coupled to one another through one or more intermediate elements.

Dental and surgical navigation systems can be particularly helpful in guiding surgical and dental interventions. These navigations systems may allow the position and orientation (the pose) of a surgical instrument (such as a dental drill) to be tracked. The tracked pose can then be compared to and aligned with the position and orientation of a region of interest in the patient's anatomy. The results of the tracking may be provided to a surgeon or dentist as visual feedback to guide the surgical intervention. This may be particularly useful when surgical instruments are used in regions of a patient's anatomy that are obscured from the surgeon's or dentist's view.

Typically, surgical instruments such as dental handpieces are not directly trackable by a pose tracking device. As a result, a special trackable object (referred to herein as a “trackable target”) may be used to track the position of the surgical instrument. Various tracking systems can be used with different forms of trackable targets. In the case of an optical tracking system, the trackable target can be marked with optical targets for the tracking camera. In the case of an electro-magnetic tracking system, the trackable target can contain magnetic sensors or a magnetic field source. In general, the trackable target must be rigidly attached to a surgical instrument to provide consistent tracking results.

For example, U.S. patent application Ser. No. 13/884,128 titled “Method and apparatus for automated registration and pose tracking” by Dekel et al, filed Nov. 23, 2011, incorporated herein by reference in its entirety, describes a medical therapy guidance system in which the tip of a tool can be mapped to a volumetric image of an anatomical region registered with the actual anatomical region in which therapy is being performed. To perform this mapping, a special trackable target can be clamped to the handle of the tool, and the tip's pose (location and orientation) relative to the trackable target can be measured and stored (calibrated). The guidance system can track the pose of the target, and, using the results of the tip calibration, map the target's pose to the tip's pose.

If a consistent spatial relationship between the target and the surgical instrument is not maintained, the results from the pose tracking systems may be inaccurate. Accordingly, the adapter used to couple the target to the surgical instrument should be substantially rigid (at least in part) and able to resist relative displacement with respect to the surgical instrument. However, a number of challenges may arise when coupling a trackable target to a surgical instrument. In some cases, the handle of a surgical instrument (e.g. a dental drill) may have a variable diameter. For example, the handle may be tapered. As a result, standard hose clamping mechanisms may easily slip and lose grip when attached to the handle of the surgical instrument.

As well, different surgical instruments, or surgical instruments from different manufacturers, may have different handle shapes or sizes. In such cases, different adapters may be used to accommodate the different shapes, sizes and taper angles commonly used in surgical instrument handles. A single adapter that is able to accommodate different shapes and sizes of surgical instruments may minimize the number of adapters a surgeon or dentist needs for the different tools they use. As well, the use of a single adapter suitable for multiple instruments may tend to make installation of the adapter (and therefore the target) simpler because the dentist or surgeon (or other medical professional) may not need to identify the correct adapter for each instrument.

Furthermore, to simplify and speed up preparations for surgery, it may be desirable to minimize disassembly and reassembly operations between operations while allowing for a thorough disinfection and sterilization of the reusable parts. Furthermore, it may be desirable for the adapter to be used with a surgical instrument and then removed without damaging the instrument's outer appearance. Embodiments described herein may alleviate or overcome some or all of the above-noted challenges.

The various embodiments described herein generally relate to a surgical instrument tracking assembly. In particular, the embodiments described herein relate to a surgical instrument tracking assembly having a trackable target that can be rigidly coupled to a substantially rigid adapter, and a length of stretchable tape for detachably attaching the adapter to a surgical instrument. The stretchable tape can be stretchably wound around the adapter and the surgical instrument to provide a tensile clamping force to hold the adapter against the surgical instrument in a fixed, stable, spatial relationship.

The trackable target and adapter may be referred to as a trackable assembly. The trackable target can be rigidly coupled to the substantially rigid adapter. When the trackable target is rigidly coupled to the adapter, the pose of the adapter can be determined from the pose of the trackable target. When the adapter is held against the surgical instrument in the fixed, stable, spatial relationship, a pose of the surgical instrument may be determinable from the pose of the adapter (which is in turn determinable from the pose of the trackable target when the trackable target is attached to the adapter). The trackable target may be trackable by a tracking device, such as an optical or electro-magnetic tracking device, to determine a pose of the trackable target.

The trackable assembly may also include at least one coupler for rigidly coupling the adapter and the trackable target. The at least one coupler can couple the trackable target and the adapter together such that the pose of the adapter is determinable from the pose of the trackable target. The at least one coupler may allow the adapter and the trackable target to be easily and quickly attached and detached from one another, with the adapter and the trackable target being secured by the at least one coupler in a consistent, stable, spatial relationship when attached.

The stretchable tape used to hold the adapter against the surgical instrument can be highly elastic. The elasticity of the stretchable tape may provide a long-lasting tensile force for holding the adapter against the surgical instrument. The stretchable tape can be selected to have an elasticity that preserves its tensile force when applied around the adapter and the surgical instrument.

The stretchable tape can also be selected to have sufficient strength to resist tearing when being stretched by a pulling force (such as a pulling force provided by a human hand stretching the stretchable tape) as the length of the stretchable tape is wound or wrapped around the adapter and the surgical instrument. This can help prevent tearing of the stretchable tape when being applied to hold the adapter against the surgical instrument. The tensile clamping force of the stretchable tape may reduce the likelihood of the adapter shifting or being detached when the surgical instrument is in use.

The stretchable tape can be self-engaging such that once the tape is wound on itself while being stretched by a pulling force, the long-lasting tensile clamping force of the winding can be maintained even when the pull force is removed. In some cases, the tensile force of the tape may be increasable from one winding of the tape around the adapter and the surgical instrument to the next winding of the length of stretchable tape around the adapter and the surgical instrument.

In some embodiments, the stretchable tape may be a self-amalgamating or self-fusing tape. A self-amalgamating stretchable tape may combine or unite with itself when it comes into contact with itself without requiring adhesive. For example, a self-fusing silicone tape may be used. In some cases, other self-amalgamating tapes such as PIB (Polyisobutylene) rubber or EPR (Ethylene Propylene) rubber tapes may be used.

In some embodiments, a stretchable adhesive tape or adhesive strip may be used. For example, the self-engaging stretchable tape may take the form of single-use pre-sterilized adhesive strips that can be wound around the adapter and the surgical instrument.

The use of a stretchable self-engaging tape may simplify the process of securing the adapter to a surgical instrument. The self-engaging tape can secure the adapter to the surgical instrument with fewer windings and provide a strong and consistent tensile clamping force to hold the adapter against the surgical instrument in a fixed, stable, spatial relationship. As well, a self-amalgamating tape can secure the adapter to the surgical instrument without being tacky. This can reduce, or eliminate, residue left on the surface of the surgical instrument from the stretchable tape when the tape is removed from the instrument.

The low-profile of the self-engaging tape around the adapter can provide a smooth and ergonomic surface that makes it easier to grasp the surgical instrument with the adapter attached thereto. In some embodiments, this low-profile may be further enhanced using a rubber cover sleeve to cover the portions of the adapter and surgical instrument having stretchable tape wrapped there around. As well, the low-profile of the self-engaging tape can reduce the possibility of the tape accidentally catching and being loosened, released or severed. For example, the smooth profile of the self-engaging tape may reduce the likelihood of the tape being accidentally released or broken when the surgical instrument is in use, such as when the surgical instrument is inserted into a cleavage.

The adapter can be used to simply, and easily, attach the trackable target to surgical instruments of various shapes and sizes. Where at least one coupler is used to rigidly and detachably attach the trackable target to the adapter, the trackable target may be detached from the adapter as required. For example, in some embodiments the adapter may be constructed from materials able to withstand sterilization processes, and repeated sterilization cycles. Similarly, the stretchable tape may also be constructed of materials capable of withstanding repeated sterilization cycles. Accordingly, the adapter may be left attached to the surgical instrument and sterilized along with the surgical instrument.

Example sterilization cycles may include steam sterilization cycles or chemical sterilization cycles for medical instruments known in the art. In various embodiments, the adapter and/or the stretchable tape may be constructed of materials capable of withstanding chemical sterilization of up to 10 hours with commonly used chemical sterilants such as hydrogen peroxide, glutaraldehyde, and OPA (ortho-phthalaldehyde) for example, without significantly modifying the properties of those materials.

The at least one coupler may then allow a single-use trackable target to be quickly attached to the adapter prior to use, then quickly discarded afterward. In embodiments where a reusable trackable target is used, the coupler may enable cleaning and disinfecting of the trackable target separately from the surgical instrument as needed. The coupler may be configured to disengage when the trackable target is accidentally hit, for example if the surgical instrument, with the trackable target attached, is accidentally dropped on a floor. The ability of the coupler to disengage may reduce the likelihood of accidental damage to either the trackable target or the surgical instrument.

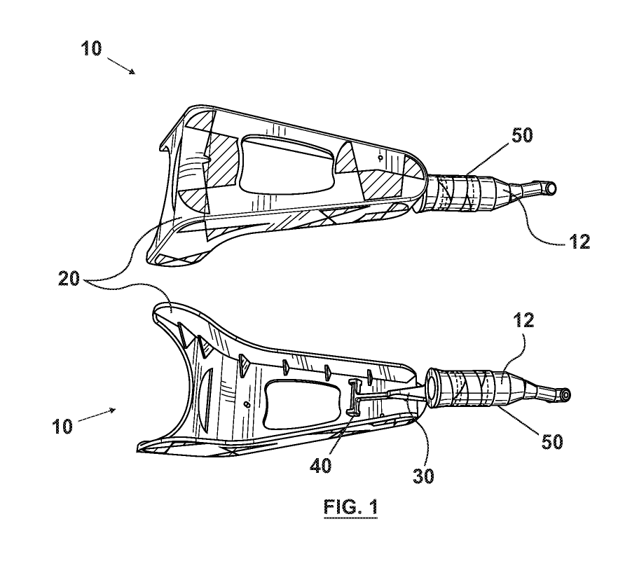

Referring now to FIG. 1, shown therein is a pair of perspective views of a surgical instrument tracking assembly 10. The surgical instrument tracking assembly 10 is an example of a tracking assembly that may be used to track the position and orientation of a surgical instrument, such as surgical instrument 12. The surgical instrument 12 shown in FIG. 1 is an example of a dental handpiece instrument that may be used by a dentist when performing oral surgery on a patient.

The surgical instrument tracking assembly 10 has a trackable assembly that includes a trackable target 20. The trackable assembly can also include a substantially rigid adapter 30. The trackable target 20 can be mounted to the surgical instrument 12 using the adapter 30. The adapter 30 can be rigidly, and detachably attached to surgical instrument 12 as will be described in further detail herein below. When desired, the adapter 30 may also be detached from the surgical instrument 12 and subsequently re-attached or attached to an alternate surgical instrument.

The trackable target 20 can be tracked by a tracking device to determine a pose of the trackable target 20. When the trackable assembly is mounted to the surgical instrument 12, the pose of the surgical instrument 12 may be determined from the pose of the trackable target 20. The trackable target 20 may be tracked using various tracking systems such as optical or electro-magnetic tracking systems.

In some embodiments, the trackable target 20 may define an optically detectable pattern detectable by a tracking device to determine the pose of the trackable target. For example, the optically detectable pattern may include a plurality of high contrast regions. The high contrast regions may be trackable by an optical tracking system such as the MicronTracker optical tracking system (ClaroNav Inc., Toronto, Canada). The trackable target 20 may include other detectable regions, for example using magnetic sensors or magnetic field sources when an electro-magnetic tracking system is used.

The adapter 30 may simplify the process of attaching and detaching a trackable target 20 to and from the surgical instrument 12. For example, the adapter 30 may be used to attach the trackable target 20 to surgical instruments having different shapes or sizes.

The adapter 30 may also allow the trackable target 20 to be easily attached to the surgical instrument 12 before a procedure and then detached when the procedure complete. For example, the adapter 30 may allow the trackable target 20 to be easily detached from the surgical instrument 12, when the surgical instrument 12 is being sterilized.

In some embodiments, the trackable assembly may include the trackable target 20 and adapter 30 as separate components, as is shown in FIG. 1. The trackable assembly can also include at least one coupler 40 for rigidly coupling the adapter 30 and the trackable target 20. When the adapter 30 and trackable target 20 are rigidly coupled, the pose of the adapter 30 may be determinable from the pose of the trackable target 20.

In various embodiments, the coupler 40 may be mounted on at least one of the adapter 30 and the trackable target 20. The coupler 40 may be releasable to allow the trackable target 20 to be detached from the adapter 30. In some embodiments, the coupler 40 may be a quick-connector coupler. For example, the coupler 40 may include mating quick-connector portions mounted on each of the adapter 30 and trackable target 20. In some cases, the coupler 40 may include a separate component with mating coupling portions provided by each of the adapter 30 and the trackable target 20. Various other coupling mechanisms for coupler 40 will be apparent to those skilled in the art.

In some embodiments, the trackable assembly may include an adapter, such as adapter 30, formed integrally with a trackable target such as trackable target 20. In such embodiments, the trackable target 20 may be attached directly to the surgical instrument 12 using the adapter.

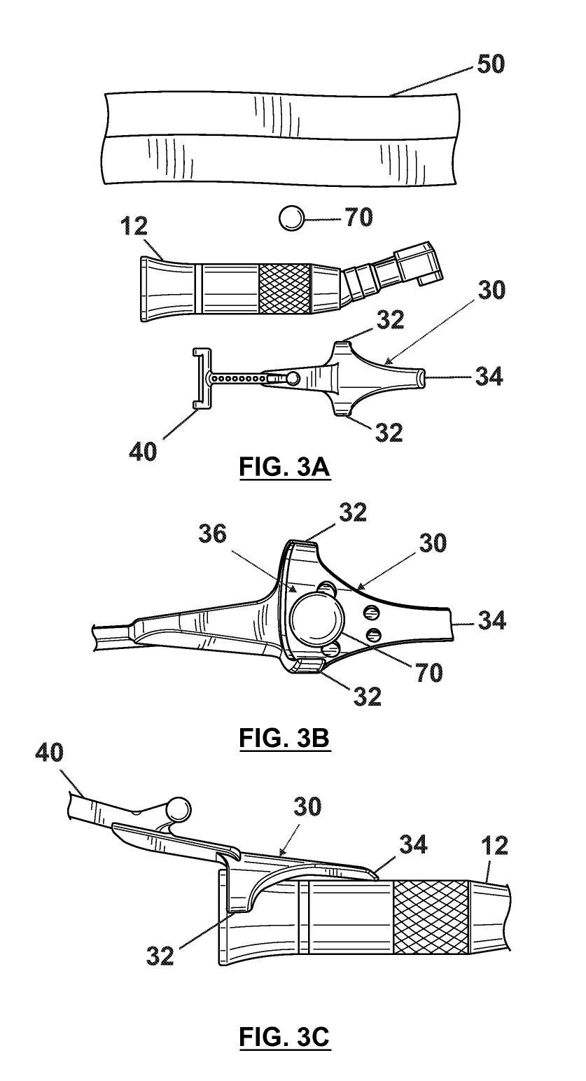

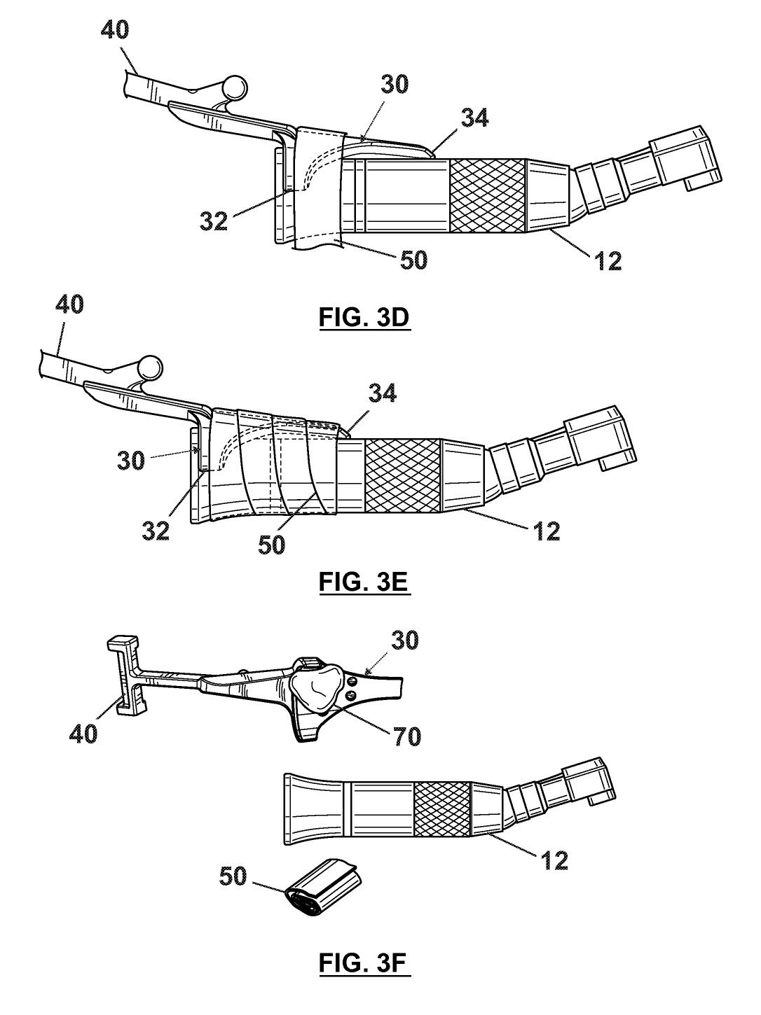

The surgical instrument tracking assembly 10 also includes a length of stretchable tape 50. The stretchable tape 50 is shown wrapped around the surgical instrument 12. The stretchable tape 50 can be used for stretchably wrapping around the adapter 20 and the surgical instrument 12 to provide a tensile force holding the adapter 30 against the surgical instrument 12 in a fixed, stable, spatial relationship (see FIGS. 3D-3E below). The adapter 30 and the trackable target 20 can be subsequently detachable from the surgical instrument 12 by removing the stretchable tape 50 (see FIG. 3F below). An example process for attaching the adapter 30 to the surgical instrument 12, and subsequently detaching the adapter 30 from the surgical instrument 12, will be described in further detail below with reference to FIGS. 3A-3F.

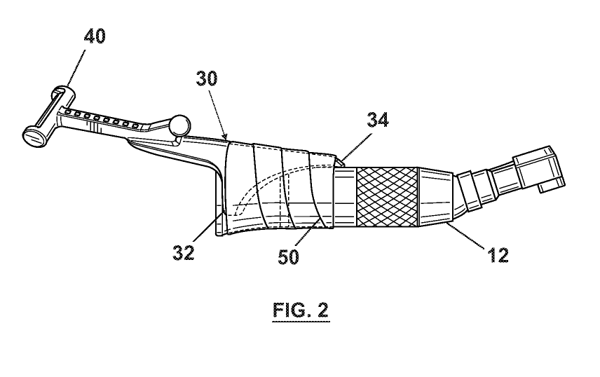

Referring now to FIG. 2, shown therein is a side view of an adapter 30 held against a surgical instrument 12 using a length of stretchable tape 50. Multiple windings of the stretchable tape 50 can be wrapped around the adapter 30 and the surgical instrument 12 to hold the adapter 30 against the surgical instrument 12 in a fixed, stable, spatial relationship. The stretchable tape 50 can be tightly wound against the adapter 30 and the surgical instrument 12 to provide a tensile force that holds the adapter 30 and the surgical instrument 12 together and resists relative displacement between the adapter 30 and the surgical instrument 12.

The adapter 30 may be constructed of materials that can withstand repeated steam-sterilization cycles or chemical sterilization cycles without distorting or losing its strength. The cycles of steam sterilization may include high pressure steam with temperatures greater than 120° C. for durations of greater than 5 minutes. For example, the adapter 30 may be constructed to withstand repeated steam-sterilization cycles using high pressure steam of up to 135° C. for durations up to 15 minutes.

In some embodiments, the adapter 30 may be constructed from corrosive resistant strong materials selected from a group including stainless steel and engineered plastic. Examples of engineered plastic may include such as or glass fiber reinforced PEEK.

The length of stretchable tape 50 can be configured to be highly elastic to provide a long-lasting tensile clamping force for holding the adapter 30 against the surgical instrument 12. The stretchable tape 50 can also be configured to be sufficiently strong to resist tearing when being stretched by a pulling force, such as a pulling force provided by one human hand, as the length of stretchable tape 50 is wrapped around the adapter 30 and the surgical instrument 12. In some embodiments, the stretchable tape 50 may be sufficiently strong to resist tearing when being stretched by a pulling force of at least about 300 N.

The stretchable tape 50 may also be self-engaging such that once the tape is wound on itself while being stretched by a pulling force, the long-lasting tensile clamping force of the winding can be maintained even when the pull force is removed. In some cases, the long-lasting tensile clamping force can be easily increased from one winding of the length of stretchable tape 50 around the adapter 30 and the surgical instrument 12 to the next winding of the length of stretchable tape 50 around the adapter 30 and the surgical instrument 12.

In some cases, the stretchable tape 50 may self-amalgamate. That is, the stretchable tape 50 may combine or unite with itself when it comes into contact with itself, e.g. when it is wrapped over itself with each winding. The windings of the stretchable tape 50 can then combine to form a substantially seamless tape layer around the adapter 30 and the surgical instrument 12. Tensile force provided by stretching the stretchable tape 50 can thus secure the adapter 30 against the surgical instrument 12 in a stable spatial relationship.

In some embodiments, the stretchable tape 50 may also be heat and/or chemical resistant to withstand repeated cycles of steam sterilization or chemical sterilization. The cycles of steam sterilization may include high pressure steam with temperatures greater than 120° C. for durations of greater than 5 minutes. For example, the steam sterilization cycles may use high pressure steam of up to 135° C. for periods up to 15 minutes in duration. This may allow the adapter 30 to be maintained against the surgical instrument 12 when the instrument is sterilized after use.

In some embodiments, the stretchable tape 50 may be a self-fusing or self-amalgamating tape such as a self-fusing silicone tape. An example of self-fusing silicone tape is available as “Miracle Wrap” under the brand name Tommy Tape, manufactured by Midsun Specialty Products (Berlin, Conn., USA).

FIG. 2 also shows a coupler 40 mounted to the adapter 30. The coupler 40 can be used to rigidly couple the adapter 30 to a trackable target. The trackable target may also include a mating coupler portion for receiving the coupler 40. A pose of the surgical instrument 12 may be determinable from the pose of the adapter 30 and from the pose of the trackable target when the trackable target is attached to the adapter 30, e.g. using the coupler 40. In some embodiments, the coupler 40 may also be constructed of sterilizable materials, allowing the adapter 30 with coupler 40 (or at least the adapter-side mating portion of coupler 40) mounted thereon to remain attached to the surgical instrument 12 when being sterilized.

Reference will now be made to FIGS. 3A-3F to describe in greater detail various components used in embodiments of a surgical instrument tracking assembly. An example process for mounting a trackable assembly to a surgical instrument will also be described with reference to FIGS. 3A-3F.

Referring now to FIG. 3A, shown therein is a top view of various components that may be used with a surgical instrument tracking assembly such as surgical instrument tracking assembly 10. The components include the surgical instrument 12, shown as a dental handpiece in this example.

The components also include the adapter 30. The adapter 30 can include a plurality of possible contact regions spaced apart from each other. The possible contact regions can be configured such that the adapter 30 is orientable relative to the surgical instrument 12 so that at least three contact regions in the plurality of possible contact regions are in contact with the surgical instrument 12.

The surgical instrument 12 may have a substantially cylindrical circumferential surface. The at least three contact regions of the adapter 30 can be oriented to contact the substantially cylindrical surface of the surgical instrument 12. In some cases, the at least three contact regions may include at least four contact regions for contacting the substantially cylindrical surface of the surgical instrument 12.

The adapter 30 may include a plurality of arms. For example, the adapter 30 includes side arms 32 and front arm 34. The plurality of possible contact regions may include a plurality of arm contact regions where each arm of the plurality of arms has a corresponding arm contact point in the plurality of arm contact regions. The plurality of arm contact regions may be oriented to contact the surface of the surgical instrument 12 when the adapter 30 is positioned to contact the surgical instrument 12.

In general, the arm contact point for each arm does not necessarily refer to a single point on that arm. For example, depending on the circumference of the surgical instrument 12, the arm contact point (or contact points) for each arm may change. For example, where the circumference of the surgical instrument 12 is greater, the arm contact point(s) for each arm may be located closer to the distal end of each arm, whereas when the circumference is smaller, the arm contact point may be located closed to the central portion of the adapter 30 (i.e. further from the distal ends).

While the adapter can be substantially rigid to maintain a fixed spatial relationship between trackable target 20 and surgical instrument 12, in some embodiments, the adapter 30 may include softer deformable or flexible portions that surround (and may include) the contact regions. The deformable portions may be configured to conform to a surface of the surgical instrument when pressed against that surface by the tensile clamping force of the stretchable tape. The deformable portions may be resiliently deformable, and may return to their initial configuration absent the tensile clamping force of the stretchable tape. As well, the deformable portions may be secured in position against the surgical instrument, and prevented from further deforming, by the tensile clamping force of the stretchable tape.

The deformable portions of the adapter 30 may include adapter portions that are thinner than the rest of the adapter to allow the contact portions to be thin and/or soft enough to deform under the force of the tape 50 pushing the adapter 30 against the surface of surgical instrument 12. The deformable portions may enable the adapter 30 to better conform to the different surface shapes of different surgical instruments. The deformable portions may also minimize the protrusion of the adapter 30 above the surgical instrument's surface. For example, in the embodiments shown in FIGS. 1-3, while the adapter can be constructed of a generally rigid material, such as stainless steel or aluminum, the tips or distal regions of arms 32 and 34 (the deformable portions) can be designed to be thin enough to become compliant with the underlying instrument surface under the clamping force of stretched tape 50. That is, the rigid portion and the at least one deformable portion of the adapter 30 can be constructed from the same material with the rigid portion of the adapter 30 being thicker than each deformable portion.

In other embodiments, two different materials may be used in the construction of the adapter 30—one rigid material to form the rigid portion of the adapter as a strong and stable shell, or spine, and one softer material to form compliant, high friction, deformable contact regions that can resist displacement and better protect the surface finish of the surgical instrument. For example, in the embodiment illustrated in FIG. 3, thin rubber pads may be firmly attached to the contact regions of arms 32 and 34 on the side facing the instrument to provide the deformable portions of adapter 30. An example of a suitable material for such pads is Sugru, made by Sugru Inc. of Livornia, Mich., USA.

In some embodiments, the at least one coupler 40 for rigidly coupling the adapter 30 and a trackable target such as trackable target 20, can be mounted on at least one of the trackable target and the adapter 30. FIG. 3A shows an example of the coupler 40 mounted to the adapter 30. The at least one coupler 40 can be releasable to detach the trackable target from the adapter 30. The at least one coupler 40 may also include a mating portion mounted on the trackable target, such as a quick-connect female connector that mates with the quick-connect male connector provided by coupler 40.

A length of stretchable tape 50 is also shown prior to being wrapped around the surgical instrument 12 and adapter 30. The desired length of stretchable tape 50 may be determined depending on the diameter of the surgical instrument. For example, for a typical dental handpiece, a 12 cm length of stretchable tape 50 may be used.

In some embodiments, sticky putty 70 may be applied between the adapter 30 and surgical instrument 12 prior to wrapping the stretchable tape 50 around the adapter 30 and the surgical instrument 12. The sticky putty 70, such as “tac ProPower” made by UHU Gmbh (www.uhu.com), may be inserted between the adapter 30 and the surgical instrument 12 to provide additional adhesion therebetween. This adhesion may be particularly useful when installing the adapter 30 on the surgical instrument 12. In FIG. 3A, a ball of sticky putty 70 is shown prior to a process for combining the elements of a surgical instrument tracking assembly.

Referring now to FIG. 3B, shown therein is a perspective view of the underside of adapter 30 with sticky putty 70 applied thereto. The sticky putty 70 has been applied to the underside of the adapter 30 prior to attaching the adapter 30 to the surgical instrument 12.

The adapter 30 may include a central portion 36 defining a radius of curvature. The plurality of arms of the adapter 30 include two side arms 32 extending from the central portion 36, and a forward arm 34 extending from the central portion 36 in a forward direction 38 substantially orthogonal to a direction of the radius of curvature.

The central portion 36 and the two side arms 32 can together define a substantially concave shape. In some embodiments, the substantially concave shape may define a diameter of about 20 mm. A 20mm diameter may optimally match the proximal end of low-speed dental handpieces designed to connect to a cylindrical motor of a 20 mm diameter. In the embodiment shown in FIG. 3, the adapter 30 has a substantially triangular shape defined by the central portion 36, the side arms 32 and the front arm 34.

In some cases, the substantially cylindrical circumferential surface of the surgical instrument 12 may be tapered. The diameter of surgical instrument 12 may vary along at least a portion of a length of the surgical instrument 12 to provide a substantially frustro-conical circumferential surface. The substantially concave shape defined by the central portion 36 and the two side arms 32 can correspond to the tapered surface of the handle of surgical instrument 12. The plurality of possible contact regions can include a central contact point within the central portion. In FIG. 3B, the stick putty 70 has been applied to the central contact point to provide adhesion between the adapter 30 and the surgical instrument 12 when the adapter 30 is positioned to contact the surgical instrument 12. Optionally, the stick putty 70 may also secure the surgical instrument 12 within the substantially concave shape defined by the central portion 36 by occupying much of the space between the central portion 36 and the surgical instrument 12. This may further obstruct or prevent relative movement between the surgical instrument 12 and the adapter 30.

Referring now to FIG. 3C, shown therein is a side view of the adapter 30 positioned to contact the surgical instrument 12. The adapter 30 can be positioned with at least three of the contract regions in contact with the surface of the surgical instrument 12 prior to wrapping the stretchable tape 50 around the adapter 30 and the surgical instrument 12.

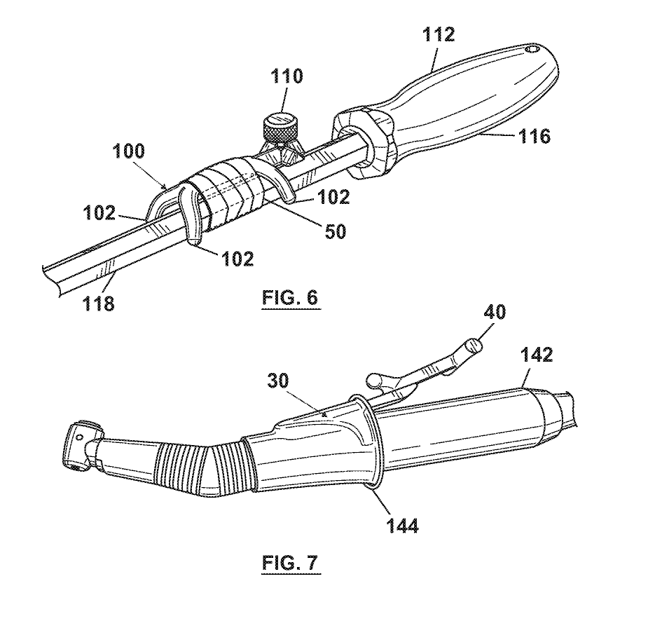

In some embodiments, such as FIG. 3C, the adapter 30 can be positioned to contact the handle of surgical instrument 12. For example, the adapter 30 (with or without sticky putty 70 in the central portion 36) can be centered on the surgical instrument 12. The adapter 30 may be centered so that the forward direction 38 is aligned with the length of the handle of the surgical instrument 12. The forward arm 34 may also be positioned to face away from the end, and towards the stem, of the surgical instrument 12. In some embodiments, such as the embodiment shown in FIG. 6, the adapter may be positioned to contact the stem of a surgical instrument 12.

After being placed in the desired position to contact the surgical instrument 12, the adapter 30 can be pressed against the surface of the surgical instrument 12. The sticky putty 70 may then provide some initial adhesion to hold the adapter 30 against the surgical instrument 12. This initial adhesion can maintain the adapter 30 in the desired position against the surgical instrument 12 as the stretchable tape 50 is wound around the adapter 30 and the surgical instrument 12.

Referring now to FIG. 3D, shown therein is a side view of the adapter 30 and the surgical instrument 12. To begin securing the adapter 30 to the surgical instrument 12, the stretchable tape 50 can be pressed against the bottom of the surgical instrument 12, near the widest portion of the adapter 30. For example, exposed end of self-fusing silicone tape can be pressed against the surgical instrument 12.

An initial winding of stretchable tape 50 is shown wrapped around the adapter 30 and the surgical instrument 12. Once a full winding of the self-engaging stretchable tape 50 is made, the stretchable tape 50 can engage with itself (e.g. adhere or amalgamate or fuse), in effect forming a strap clamping the adapter 30 against the surgical instrument 12.

Referring now to FIG. 3E, shown therein is a side view of the adapter 30 and the surgical instrument 12 with multiple windings of the stretchable tape 50. The stretchable tape 50 has been wound around the adapter 30 and the surgical instrument 12. The stretchable tape 50 generally covers the portion of the adapter 30 positioned to contact the surgical instrument 12. Because the tape 50 can self-engage when wound around itself, the clamping force of prior windings can be maintained even when the user momentarily reduces the pulling force on the tape during the winding procedure. Each winding of the stretchable tape 50 thus generally adds to the prior clamping force holding the adapter 30 against the surgical instrument 12. Accordingly, with a sufficient number of forceful windings by hand, typically in the range of 5-8, the stretchable tape 50 can clamp the adapter 30 against the surgical instrument 12 with sufficient force to ensure a fixed, stable spatial relationship throughout the surgery.

The adherence and/or high friction between the tape 50 and the surface of the surgical instrument 12 on the side opposite to the surface against which the adapter 30 is held further assists in retaining the adapter 30 in position. This can prevent the tape 50 from shifting relative to the surgical instrument 12, and with similar adherence or friction between the adapter 30 and tape 50 provided by the winding, the adapter 30 can be further retained in the fixed, stable relationship with the surgical instrument 12. This may be particularly important when the adapter 30 is mounted on a tapered surgical instrument, where the clamping force may be reduced when the adapter 30 is momentarily pushed in the tapering direction.

To ensure that the fixed, stable, spatial relationship has been established, no movement or slack should be felt when manually attempting to move the adapter 30 relative to the surgical instrument 12. If there is slack or relative movement between the adapter 30 and the surgical instrument 12, additional windings with an additional piece of stretchable tape 50 may be added to increase the clamping force. Alternatively, the stretchable tape 50 can be easily removed by cutting it (as shown in FIG. 3F), and the adapter 30 can be re-applied to the surgical instrument 12 to ensure the proper stable relationship is established.

The self-engaging stretchable tape 50 also allows the adapter 30 to be easily secured to the surgical instrument 12. With the adapter 30 initially held in place against the surgical instrument 12 (e.g. using sticky putty 70), the stretchable tape 50 can be easily wound around the adapter 30 and surgical instrument 12. This allows a quick and easy installation of the adapter 30 against the surgical instrument 12.

Referring now to FIG. 3F, shown therein is a perspective view of the components of the surgical instrument tracking assembly after the adapter 30 has been detached from the surgical instrument 12. The adapter 30 may be detached from the surgical instrument 12 for use with a different surgical instrument, or if slack or relative movement between the adapter 30 and surgical instrument 12 is detected, the adapter 30 may be detached from and subsequently reattached to the surgical instrument 12. In some cases, the adapter 30 may be removed for sterilization of the various components of the surgical instrument tracking assembly.

The adapter 30 may be detached by removing the stretchable tape 50 from the surgical instrument 12. For example, the stretchable tape 50 may be severed using a knife. Once the stretchable tape 50 has been removed, the adapter 30 can be removed from the surgical instrument 12. The process of installing the adapter 30 on a surgical instrument 12 can then be repeated with surgical instrument 12 or a different surgical instrument.

After the removable of the stretchable tape 50 and adapter 30, no residual marks may be left on surgical instrument 12. In some cases, this may be due to the use of a non-tacky stretchable tape 50. The sticky putty 70 may also be subsequently removed from the underside of the adapter 30.

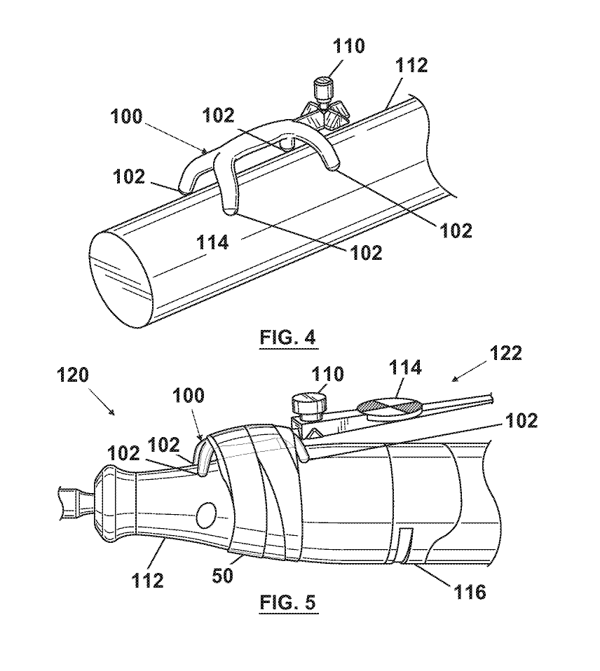

Referring now to FIG. 4, shown therein is a perspective view of an alternative embodiment of an adapter 100 positioned to contact a surgical instrument 112. The portion of surgical instrument 112 shown in FIG. 4 (the stem) has a substantially cylindrical circumferential shape. The plurality of arms of adapter 100 includes four arms 102 that may contact the surface of surgical instrument 112. In this example embodiment, the plurality of arm contact regions comprises four arm contact regions, including a corresponding arm contact point for each arm 102 of the four arms 102.

The four arm contact regions, corresponding to arms 102, may be orientable to simultaneously contact the substantially cylindrical surface of the surgical instrument 112. The inner surface of each arm 102 may correspond to an arm contact region. For example, when oriented along the length of the surgical instrument 112, all four arm contact regions may simultaneously contact the surface of the surgical instrument. As mentioned above, the corresponding arm contact points for each arm 102 may depend on the diameter of the particular surgical instrument. Embodiments of the adapter 100 may also be suitable for frustro-conical surgical instruments in which the cylindrical surface is tapered as the diameter of the surgical instrument changes along its length, as described below with reference to FIG. 5.

The adapter 100 can also include a coupler 110 mounted thereto. The coupler 110 may be used to couple a trackable target to the adapter 100. The coupler 110 is another example of a quick-release coupler that can be coupled to a quick-release coupler mating portion mounted on the trackable target.

Referring now to FIG. 5, shown therein is a perspective view of another example of a surgical instrument tracking assembly 120. The surgical instrument tracking assembly 120 includes a trackable assembly 122. The tracking assembly 122 includes an adapter 100 coupled to a trackable target 114 using a coupler 110. The surgical instrument tracking assembly 120 also includes a length of stretchable tape 50.

The handle 116 of surgical instrument 112 still has a substantially cylindrical circumferential surface. However, the handle 116 has a varying diameter such that the surface of the handle 116 is tapered. Thus, the surgical instrument 112 has a substantially frustro-conical circumferential surface. Nonetheless, the arm contact regions provided on the inner surface of each 102 are able to contact the surface of the handle 116.

The adapter 100 is shown contacting the surgical instrument 112 with the length of stretchable tape 50 wrapped around the adapter 100 and the handle 116 of the surgical instrument 112. The stretchable tape 50 can hold the adapter 100 against the surgical instrument 112 in a fixed, stable, spatial relationship. As mentioned above, the stretchable tape 50 may provide a tensile clamping force holding the arm contact regions in a static position of the surface of the handle 116 and resisting relative displacement between the adapter 100 and the surgical instrument 112.

Referring now to FIG. 6, shown therein is a perspective view of the adapter 100 positioned to contact the stem 118 of the surgical instrument 112. Here, the stem 118 has a substantially cylindrical shape with a smaller diameter than the handle 116. Accordingly, different arm contact points within the arm contact regions corresponding to each arm 102 may be in contact with the surgical instrument 112. Once again, a length of stretchable tape 50 can be used to hold the adapter 100 against the surgical instrument 112.

Referring now to FIG. 7, shown therein is an example embodiment of a rubber cover sleeve 144 that may be used with embodiments of a surgical instrument tracking assembly, such as the surgical instrument tracking assemblies 10 and 120. The rubber cover sleeve 144 can be configured to engage the adapter 30 and the surgical instrument 142 when attached together to cover the length of stretchable tape used to hold the adapter 30 against the surgical instrument 142.

The rubber cover sleeve 144 may also be manufactured from materials that can withstand repeated steam-sterilization cycles without distortion. This may allow the rubber cover sleeve 144 to be maintained in place when the surgical instrument 142 is sterilized.

The rubber cover sleeve 144 may provide a smoother instrument surface for the surgical instrument 142 when in use. As well, the rubber cover sleeve 144 may obscure the adapter 30 and stretchable tape 50 to provide a more sleek and sterile appearance to a patient when the surgical instrument 142 is in use. This may reassure the patient that the surgical instrument 142 is sterile. Various sizes of rubber cover sleeve 144 may be used depending on the diameter of surgical instrument 142.

Various embodiments have been described herein by way of example only. Various modification and variations may be made to these example embodiments without departing from the spirit and scope of the invention, which is limited only by the appended claims.

1. A surgical instrument tracking assembly comprising:
a trackable assembly comprising a trackable target, the trackable target being trackable by a tracking device to determine a pose of the trackable target, and an adapter rigidly coupleable to the trackable target such that a pose of the adapter is determinable from the pose of the trackable target; and
a length of stretchable tape for stretchably winding around the adapter and a surgical instrument to provide a tensile clamping force holding the adapter to the surgical instrument in a fixed, stable, spatial relationship, the adapter and trackable target being subsequently detachable by removing the stretchable tape;
wherein,
a pose of the surgical instrument is determinable from the pose of the adapter and from the pose of the trackable target when the trackable target is attached to the adapter; and,
the length of stretchable tape is configured to be i) highly elastic to provide a long-lasting tensile clamping force for holding the adapter against the surgical instrument, ii) sufficiently strong to resist tearing when being stretched by a pulling force of one human hand as the length of stretchable tape is wound around the adapter and the surgical instrument, and iii) self-engaging such that once the tape is wound on itself while being stretched by the pulling force, the long-lasting tensile clamping force of the winding is maintained even when the pulling force is removed.

2. The surgical instrument tracking assembly as defined in claim 1 wherein the tape is self-fusing.

3. The surgical instrument tracking assembly as defined in claim 1 wherein the tape is self-fusing silicone tape.

4. The surgical instrument tracking assembly as defined in claim 1 wherein the surgical instrument is a dental handpiece.

5. The surgical instrument tracking assembly as defined in claim 1 further comprising sticky putty for applying between the adapter and the surgical instrument prior to winding the length of stretchable tape around the adapter and the surgical instrument.

6. The surgical instrument tracking assembly as defined in claim 1 further comprising a rubber cover sleeve configured to engage the adapter and the surgical instrument when attached together to cover the length of stretchable tape.

7. The surgical instrument tracking assembly as defined in claim 1 wherein the adapter comprises a plurality of possible contact regions spaced from each other and configured such that the adapter is orientable relative to the surgical instrument such that at least three contact regions in the plurality of possible contact regions are in contact with the surgical instrument.

8. The surgical instrument tracking assembly as defined in claim 7 wherein the surgical instrument has a substantially cylindrical circumferential surface and the at least three contact regions are oriented to contact the substantially cylindrical surface of the surgical instrument.

9. The surgical instrument tracking assembly as defined in claim 8 wherein the at least three contact regions comprise at least four contact regions for contacting the substantially cylindrical surface of the surgical instrument.

10. The surgical instrument tracking assembly as defined in claim 8 wherein
the adapter comprises a plurality of arms,
the plurality of possible contact regions comprises a plurality of arm contact regions; and
each arm of the plurality of arms comprises a corresponding arm contact point in the plurality of arm contact regions.

11. The surgical instrument tracking assembly as defined in claim 10 wherein
the adapter further comprises a central portion defining a radius of curvature;
the plurality of arms comprises two side arms extending from the central portion, and a forward arm extending from the central portion in a forward direction substantially orthogonal to a direction of the radius of curvature;
the central portion and the two side arms together define a substantially concave shape;
the plurality of possible contact regions comprises the central contact point, the central portion comprising the central contact point.

12. The surgical instrument tracking assembly as defined in claim 10 wherein the plurality of arms comprises four arms and the plurality of arm contact regions comprises four arm contact regions, including a corresponding arm contact point for each arm of the four arms, such that the four arm contact regions are orientable to simultaneously contact the substantially cylindrical surface of the surgical instrument.

13. The surgical instrument tracking assembly as defined in claim 10 wherein the substantially concave shape defines a diameter of about 20 mm.

14. The surgical instrument tracking assembly as defined in claim 10 wherein the substantially cylindrical circumferential surface of the surgical instrument is tapered, such that a diameter of surgical instrument varies along at least a portion of a length of the surgical instrument to provide a substantially frustro-conical circumferential surface.

15. The surgical instrument tracking assembly as defined in claim 1 wherein the trackable target defines an optically detectable pattern detectable by the tracking device to determine the pose of the trackable target, the optically detectable pattern comprising a plurality of high contrast regions.

16. The surgical instrument tracking assembly as defined in claim 1 wherein the adapter is constructed from a corrosive resistant strong material selected from the group comprising stainless steel and engineered plastic.

17. The surgical instrument tracking assembly as defined in claim 7 wherein the adapter comprises a rigid portion and at least one deformable portion configured to conform to a surface of the surgical instrument when pressed against that surface by the tensile clamping force of the stretchable tape, the at least one deformable portion comprising at least one of the possible contact regions.

18. The adapter as defined in claim 17 wherein the rigid portion and the at least one deformable portion are constructed from the same material and the rigid portion of the adapter is thicker than each deformable portion.

19. The surgical instrument tracking assembly as defined in claim 1 wherein, the trackable assembly further comprises at least one coupler for rigidly coupling the adapter and the trackable target together such that a pose of the adapter is determinable from the pose of the trackable target.

20. The surgical instrument tracking assembly as defined in claim 17 wherein the at least one coupler for rigidly coupling the adapter and the trackable target is mounted on at least one of the trackable target and the adapter, the at least one coupler being releasable to detach the trackable target from the adapter.

21. The surgical instrument tracking assembly as defined in claim 1 wherein the adapter and the trackable target are formed integrally.

22. The surgical instrument tracking assembly as defined in claim 1, wherein the adapter is constructed to withstand repeated steam-sterilization cycles comprising high pressure steam up to 135° C. for 15 minutes.

23. The surgical instrument tracking assembly as defined in claim 1, wherein the length of stretchable tape is heat resistant to withstand repeated cycles of steam sterilization comprising high pressure steam up to 135° C. for 15 minutes.
